(19) United States
(12) Patent Application Publication
(10) Pub. No.: US 2023/0256131 A1
(43) Pub. Date: Aug. 17, 2023

(54) COLLAGEN-BIOGLASS ARTICLES AND METHODS OF MAKING THE SAME

(71) Applicant: 3M INNOVATIVE PROPERTIES COMPANY, St. Paul, MN (US)

(72) Inventors: Bryan A. Baker, Minneapolis, MN (US); Randilynn B. Christensen, Pine Springs, MN (US)

(21) Appl. No.: 18/012,614

(22) PCT Filed: Jun. 10, 2021
(86) PCT No.: PCT/IB2021/055133

§ 371 Date: Dec. 22, 2022

Related U.S. Application Data
(60) Provisional application No. 63/043,519, filed on Jun. 24, 2020.

(51) Int. Cl. A61L 15/18; A61L 15/32; A61L 15/42

(52) U.S. Cl. CPC A61L 15/18; A61L 15/325; A61L 15/42

(57) ABSTRACT

An article and a method of making the article. The article can include a conformable matrix comprising a mixture of collagen and bioactive glass; wherein the article is a conformable wound dressing. The method includes dissolving collagen in an acid solution; adding bioactive glass to the acid solution; mixing collagen and bioactive glass to increase or decrease the pH of the solution; and forming a mixture of collagen and bioactive glass.

### BACKGROUND

Collagen dressings are used as wound care products. These products are primarily derived from bovine collagen sources, particularly bovine skin, and processed via acid or enzymatic extraction methods into purified and largely type I collagen material.

Bioglass has been investigated and various formulations patented since the late 1960s when it was first introduced by Hench. The initial work has focused largely on bioglass, or bioactive glass, for bone tissue engineering or treatment of bone defects. Bioglass has been shown to form hydroxyapatite in the presence of simulated body fluid, and in bone tissue applications. The ionic conversion of the glass at the interface is thought to assist in the hydroxyapatite formation characteristic of bioactive glasses.

There is a need to provide better collagen dressings in a form useful for treating wounds.

### SUMMARY

Thus, in one aspect, the present disclosure provides an article. The article can include a conformable matrix comprising a mixture of collagen and bioactive glass; wherein the article is a conformable wound dressing.

In another aspect, the present disclosure provides a method of making an article. The method can include dissolving collagen in an acid solution; adding bioactive glass to the acid solution; mixing collagen and bioactive glass to increase or decrease the pH of the solution; and forming a mixture of collagen and bioactive glass.

### DETAILED DESCRIPTION

Before any embodiments of the present disclosure are explained in detail, it is understood that the invention is not limited in its application to the details of use, construction, and the arrangement of components set forth in the following description. The invention is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized and structural or logical changes may be made without departing from the scope of the present disclosure.

In some embodiments, an article suitable for use as a wound dressing is described. The article includes a conformable matrix comprising a mixture of collagen and bioactive glass. In some embodiments, the article is a conformable wound dressing. A conformable wound dressing means that the dressing can be shaped to the contours of a wound bed, enabling interaction of the dressing with a non-uniform surface found in different kinds of wounds.

The conformable matrix can be prepared by lyophilization In some embodiments, the conformable matrix is porous. The thickness of the conformable matrix is typically about 0.8 mm to about 10 mm.

In some embodiments, the weight % of bioactive glass in the conformable matrix is greater than 3 weight %, greater than 5 weight %, greater than 10 weight %, greater than 15 weight %, greater than 20 weight %, greater than 30 weight %, greater than 40 weight %, greater than 50 weight %, greater than 60 weight %, greater than 70 weight %, greater than 80 weight %, or greater than 90 weight %.

In some embodiments, the weight % of bioactive glass in the conformable matrix is less than 95 weight %, less than 90 weight %, less than 80 weight %, less than 70 weight %, less than 60 weight %, less than 50 weight %, less than 45 weight %, less than 40 weight %, less than 35 weight %, less than 30 weight %, less than 25 weight %, less than 20 weight %, less than 15 weight %, or less than 10 weight %.

In some embodiments, the weight % of bioactive glass in the conformable matrix is about 50-95 weight %. In some embodiments, the weight % of bioactive glass in the conformable matrix is about 50-70 weight %. In some embodiments, the weight % of bioactive glass in the conformable matrix is about 70-95 weight %. In some embodiments, the weight % of bioactive glass in the conformable matrix is about 60-80 weight %.

In some embodiments, the weight % of bioactive glass in the conformable matrix is about 3-50 weight %. In some embodiments, the weight % of bioactive glass in the conformable matrix is about 3-35 weight %. In some embodiments, the weight % of bioactive glass in the conformable matrix is about 3-25 weight %. In some embodiments, the weight % of bioactive glass in the conformable matrix is about 5-50 weight %. In some embodiments, the weight % of bioactive glass in the conformable matrix is about 5-35 weight %. In some embodiments, the weight % of bioactive glass in the conformable matrix is about 5-25 weight %. In some embodiments, the weight % of bioactive glass in the conformable matrix is about 10-50 weight %. In some embodiments, the weight % of bioactive glass in the conformable matrix is about 10-35 weight %. In some embodiments, the weight % of bioactive glass in the conformable matrix is about 10-25 weight %. In some embodiments, the weight % of bioactive glass in the conformable matrix is about 25-50 weight %. In some embodiments, the weight % of bioactive glass in the conformable matrix is about 35-50 weight %.

In some embodiments, the weight % of bioactive glass in the conformable matrix is about 3-10 weight %. In some embodiments, the weight % of bioactive glass in the conformable matrix is about 10-20 weight %. In some embodiments, the weight % of bioactive glass in the conformable matrix is about 20-30 weight %. In some embodiments, the weight % of bioactive glass in the conformable matrix is about 30-40 weight %. In some embodiments, the weight % of bioactive glass in the conformable matrix is about 40-50 weight %.

In some embodiments, the weight % of bioactive glass in the conformable matrix is about 5-35 weight %. In some embodiments, the weight % of bioactive glass in the conformable matrix is about 5-30 weight %. In some embodiments, the weight % of bioactive glass in the conformable matrix is about 5-15 weight %. In some embodiments, the weight % of bioactive glass in the conformable matrix is about 15-25 weight %. In some embodiments, the weight % of bioactive glass in the conformable matrix is about 25-35 weight %. In some embodiments, the weight % of bioactive glass in the conformable matrix is about 35-45 weight %.

In some embodiments, the article can include a substrate. The substrate can be selected from foam, mesh, netting, woven, nonwoven, cotton, cellulose fabrics, perforated film, hydrocolloid, hydrogel, polymers with inherent porosity, pressure sensitive adhesive and combination of thereof. In some embodiments, the substrate can be an absorbent substrate selected from foam, mesh, netting, woven, nonwoven, cotton, cellulose fabrics, perforated film, hydrocolloid, hydrogel, polymers with inherent porosity, pressure sensitive adhesive and combination of thereof. Exemplary absorbent substrate can include film, fabrics or porous article made from viscose, rayon, alginate, gauze, biopolymers, polyurethane, biodegradable polymers or the polymers described in U.S. Pat. No. 7,745,509, the disclosures of which is hereby incorporated by reference. The absorbent materials used in the absorbent substrate can be manufactured of any suitable materials including, but not limited to, woven or nonwoven cotton or rayon or netting and perforated film made from nylon, polyester or polyolefins. Absorbent pad can be used as the absorbent layer and can be useful for containing a number of substances, optionally including drugs for transdermal drug delivery, chemical indicators to monitor hormones or other substances in a patient, etc.

The absorbent layer may include a hydrocolloid composition, including the hydrocolloid compositions described in U.S. Pat. Nos. 5,622,711 and 5,633,010, the disclosures of which are hereby incorporated by reference. The hydrocolloid absorbent may comprise, for example, a natural hydrocolloid, such as pectin, gelatin, or carboxymethylcellulose (CMC) (Aqualon Corp., Wilmington, Del.), a semi-synthetic hydrocolloid, such as cross-linked carboxymethylcellulose (X4ink CMC) (e.g. Ac-Di-Sol; FMC Corp., Philadelphia, Pa.), a synthetic hydrocolloid, such as cross-linked polyacrylic acid (PAA) (e.g., CARBOPOL™ No. 974P; B.F. Goodrich, Brecksville, Ohio), or a combination thereof. Absorbent layer can be manufactured of other synthetic and natural hydrophilic materials including polymer gels and foams. In one embodiment the substrate is a hydrocolloid polymer.

The article can be in any suitable physical form, such as a sheet (i.e. film), foam sheet, or collagen-bioactive glass disposed on or within a carrier layer. For example, the conformable matrix can be disposed on or within a carrier. In some embodiments, the carrier can be a carrier layer disposed on a major surface of the article. A carrier layer is typically disposed on the opposing major surface as the wound-facing surface.

In some embodiments, carrier layer is a release liner. The release liner carrier may be disposed on the opposing major surface of both major surfaces (not shown) such that the collagen-containing sheet is between the release liner layers.

Various release liners are known such as those made of (e.g. kraft) papers, polyolefin films such as polyethylene and polypropylene, or polyester. The films are preferably coated with release agents such as fluorochemicals or silicones. For example, U.S. Pat. No. 4,472,480 describes low surface energy perfluorochemical liners. Examples of commercially available silicone coated release papers are POLYSLIK™, silicone release papers available from Rexam Release (Bedford Park, Ill.) and silicone release papers supplied by LOPAREX (Willowbrook, Ill.). Other non-limiting examples of such release liners commercially available include siliconized polyethylene terephthalate films commercially available from H. P. Smith Co. and fluoropolymer coated polyester films commercially available from 3M under the brand “ScotchPak™” release liners.

In other embodiments, the carrier layer may comprise a variety of other (e.g. flexible and/or conformable) carrier materials such as polymeric films and foams as well as various nonwoven and woven fibrous materials, such as gauze. In some embodiments, the carrier layer is absorbent, such as an absorbent foam. In other embodiments, the carrier layer is non-absorbent, such as a polymeric film.

In some embodiments, the weight ratio of collagen to bioactive glass (weight:weight) in an article is about 1:12 to about 40:1; about 1:12 to about 30:1; or about 1:12 to about 25:1.

In some embodiments, the weight ratio of collagen to bioactive glass (weight:weight) in an article is about 1:1 to about 40:1; about 1:1 to about 30:1; about 1:1 to about 25:1; about 1:1 to about 10:1; about 1:1 to about 5:1; about 1:1 to about 3:1; or about 1:1 to about 2:1.

In some embodiments, the weight ratio of collagen to bioactive glass (weight:weight) in an article is about 1.5:1 to about 40:1; about 1.5:1 to about 30:1; about 1.5:1 to about 25:1; about 1.5:1 to about 10:1; about 1.5:1 to about 5:1; or about 1.5:1 to about 3:1.

In some embodiments, the weight ratio of collagen to bioactive glass (weight:weight) in an article is about 2:1 to about 40:1; about 2:1 to about 30:1; about 2:1 to about 25:1; about 2:1 to about 10:1; about 2:1 to about 9:1

In some embodiments, the weight ratio of collagen to bioactive glass (weight:weight) in an article is about 3:1 to about 40:1; about 3:1 to about 30:1; about 3:1 to about 25:1; about 3:1 to about 10:1; about 3:1 to about 9:1; about 4:1 to about 40:1; about 4:1 to about 30:1; about 4:1 to about 25:1; about 4:1 to about 10:1; or about 4:1 to about 9:1.

The weight ratio of collagen to bioactive glass (weight:weight) in an article can range from about 1:12 to about 9.5:1. In some embodiments, the weight ratio of collagen to bioactive glass (weight\weight) in an article is from about 1:2 to about 1:12. In some embodiments, the weight ratio of collagen to bioactive glass (weight\weight) in an article is from about 1:1 to about 9.5:1. In some embodiments, the weight ratio of collagen to bioactive glass (weight\weight) in an article is from about 1:1 to about.4:1. In some embodiments, the weight ratio of collagen to bioactive glass (weight\weight) in an article is from about 4:1 to about.9.5:1.

The conductivity of the mixture of collagen and bioactive glass component of an article can be determined by Method A of the current application described herein. In some embodiments, the conductivity as determined by Method A can be less than 7, 6, 5, 4, 3, 2, 1, 0.9, 0.8, 0.7, 0.6, 0.5, 0.4, 0.3, or 0.2 mS/cm. In some embodiments, the conductivity as determined by Method A can be about 0.2-3 mS/cm. In some embodiments, the conductivity as determined by Method A can be about 0.2-2 mS/cm. In some embodiments, the conductivity as determined by Method A can be about 0.2-1 mS/cm. In some embodiments, the conductivity as determined by Method A can be about 0.5-3 mS/cm. In some embodiments, the conductivity as determined by Method A can be about 0.5-1.5 mS/cm.

Method A is described as follows: A 10 mg/mL suspension of the mixture of collagen and bioactive glass in distilled water is prepared. The water is maintained at 25° C. and the mixture of collagen and bioactive glass is completely immersed in the water. After immersion of the mixture of collagen and bioactive glass for 16 hours, the conductivity of the water is measured (mS/cm) using a conductivity meter.

In some embodiments, the conformable matrix of collagen and bioactive glass has a pH value of more than 5, 6, 7, 8, 9, or 10 as determined by Method B of the current application described herein. In some embodiments, the conformable matrix of collagen and bioactive glass has a pH value of about 6 to about 11.5 as determined by Method B. In some embodiments, the conformable matrix of collagen and bioactive glass has a pH value of about 7.5 to about 11.5 as determined by Method B. In some embodiments, the conformable matrix of collagen and bioactive glass has a pH value of about 8 to about 11.5 as determined by Method B. In some embodiments, the conformable matrix of collagen and bioactive glass has a pH value of about 6 to about 6.8 as determined by Method B.

Method B is described as follows: A 10 mg/mL suspension of the conformable matrix of collagen and bioactive glass is prepared in distilled water. The pH of the distilled water before the addition of the conformable matrix is 6.8-7.2. The water is maintained at 25° C. and the matrix of collagen and bioactive glass is completely immersed in the water. Following immersion for 24 hours, the pH value of the water is measured using a calibrated pH meter.

In some embodiments, the conformable matrix of collagen and bioactive glass has a pH value of less than 12, 11.5, 11, 10, 9, 8, or 7 determined by Method B. Not to be bound by theory, altering pH may help facilitate the recovery of wound tissue by reducing enzymatic activity

Any suitable sources of collagen can be used in the preparation of the mixture of collagen and bioactive glass. For example, the species from which the collagen is obtained could be human, bovine, porcine, or other animal sources. Collagen can also be obtained from recombinant sources. Collagen can also be obtained commercially as aqueous solutions, and the concentrations of these solutions may vary. Alternatively, collagen can be provided in lyophilized form and stored at very low temperatures. In some embodiments, collagen can be dissolved in acetic acid. In some embodiments, the amount of collagen is at least 1 mg/mL and typically no greater than 120 mg/mL.

Bioactive glass used in the invention may be melt-derived or sol-gel derived. A bioactive glass material suitable for the present articles and methods may have silica, sodium, calcium, phosphorous, and boron present, as well as combinations thereof. In some embodiments, sodium, boron, phosphorous and calcium may each be present in the compositions in an amount of about 1% to about 99%, based on the weight of the bioactive glass. In further embodiments, sodium, boron, phosphorous and calcium may each be present in the composition in about 1%, about 2%, about 3%, about 4%, about 5%, about 6%, about 7%, about 8%, about 9%, or about 10%. In certain embodiments, silica, sodium, boron, and calcium may each be present in the composition in about 5 to about 10%, about 10 to about 15%, about 15 to about 20%, about 20 to about 25%, about 25 to about 30%, about 30 to about 35%, about 35 to about 40%, about 40 to about 45%, about 45 to about 50%, about 50 to about 55%, about 55 to about 60%, about 60 to about 65%, about 65 to about 70%, about 70 to about 75%, about 75 to about 80%, about 80 to about 85%, about 85 to about 90%, about 90 to about 95%, or about 95 to about 99%. Some embodiments may contain substantially one or two of sodium, calcium, phosphorous, and boron with only traces of the other(s). The term “about” as it relates to the amount of calcium phosphate present in the composition means +/−0.5%. Thus, about 5% means 5+/−0.5%. Divalent cations or ions that may be present in any of the bioactive glasses of this and other aspects of the invention include one or more of iron-ll, iron-Ill, alumina, cobalt, copper, magnesium, and zinc. Strontium, Potassium, Fluorine. Silver, barium, titanium

The bioactive glass materials may further comprise one or more of a silicate, borosilicate, borate, or calcium, including CaO, P2O₅, SiO₂, and B₂O₃. An exemplary bioactive glass is 45S5, which includes 46.1 mol % SiO₂, 26.9 mol % CaO, 24.4 mol % Na2O and 2.5 mol % P2O₅. An exemplary borate bioactive glass is 45S5B1, in which the SiO2 of 45S5 bioactive glass is replaced by B2O3. Other exemplary bioactive glasses include 58S, which includes 60 mol % SiO2, 36 mol % CaO and 4 mol % P₂O₅, and S70C30, which includes 70 mol % SiO₂ and 30 mol % CaO. Other exemplary bioactive glasses include PSr40 which is 50% P₂O₅, 40% SrO, 10% Na₂O Mol %.

The bioactive glass may comprise one or more of SiO₂, CaO, Na₂O, P₂O₅, K₂O, MgO, and B₂O₃. The bioactive glass may comprise CaO, Na₂O, and P₂0₅.

The following composition, having a weight % of each element in oxide form in the range indicated, will provide one of several bioactive glass compositions that may be used to form a bioactive glass:

| | |
|---|---|
| SiO₂ | 0-86 |
| CaO | 4-35 |
| Na₂O | 0-35 |
| P₂O₅ | 2-15 |
| CaF₂ | 0-25 |
| B₂O₃ | 0-75 |
| K₂O | 0-8 |
| MgO | 0-5 |
| CaF | 0-35 |

In some embodiments, the conformable matrix can further comprise a plasticizing agent, for example, glycerol, diglycerol, triglycerol, xylitol, mannitol, C3-C24 alkane diols like butane diol and propane diol, to improve the hydration of the article. In addition, the plasticizing agent can improve the conformability and handleability of the article by reducing the brittleness and allowing deformation before cracking or tearing. In some embodiments, the conformable matrix can be in any suitable form, for example, a gel, sponge, scaffold, foam or film.

In some embodiments, the weight % of plasticizing agent in the conformable matrix is about 0.01 to about 1.5 weight %. In some embodiments, the weight % of plasticizing agent in the conformable matrix is about 0.01 to about 1 weight %. In some embodiments, the weight % of plasticizing agent in the conformable matrix is about 0.01 to about 0.5 weight %.

In some embodiments, the weight % of glycerol in the conformable matrix is about 0.01 to about 1.5 weight %. In some embodiments, the weight % of glycerol in the conformable matrix is about 0.01 to about 1 weight %. In some embodiments, the weight % of glycerol in the conformable matrix is about 0.01 to about 0.5 weight %.

In some embodiments, a method of forming an article of the current application is described. The method can include dissolving collagen in an acid solution, for example in acetic acid; adding bioactive glass to the acid solution; mixing collagen and bioactive glass to increase or decrease the pH of the solution;; and forming a mixture of collagen and bioactive glass. In the method, a plasticizing agent (for example glycerol) can optionally be added to the acid solution. The method may further comprise forming the mixture into a gel, sheet, film, sponge, foam, or a plurality of pieces.

In some embodiments, the method may further comprise dehydrating the mixture of collagen and bioactive glass. The dehydrating may be conducted by suitable means, such as, freeze-drying, oven drying, critical point drying, or combination thereof.

In some embodiments, the method may further comprise adding the matrix on a substrate. In some embodiments, the method may further comprise disposing the conformable matrix on or within a carrier.

In some embodiments, a method of treating a wound with an article of the current application is described. The method of treatment involves covering at least a portion of the wound with the article. In some embodiments, the method of treatment increases the pH of the wound environment. In some embodiments, the method of treatment decreases the pH of the wound environment. The wound to be treated by the method can be an open wound of the skin that exposes underlying body tissue. Open wounds that can be treated by the method include acute wounds and chronic wounds. Open wounds that can be treated by the method include wounds to the skin from trauma (for example avulsions, incisions, and lacerations); wounds to the skin from pressure (for example pressure ulcers); and wounds to the skin from disease (for example venous ulcers, diabetic foot ulcers, and diabetic leg ulcers).

The following working examples are intended to be illustrative of the present disclosure and not limiting.

### EXAMPLES

Objects and advantages of this invention are further illustrated by the following examples, but the particular materials and amounts thereof recited in these examples, as well as other conditions and details, should not be construed to unduly limit this invention.

### Materials

Collagen type I from bovine calf skin (product #C857) was obtained from Elastin Products Company, Owensville, Mo.
Glycerol was obtained from the Sigma-Aldrich Corporation, St. Louis, Mo.

Phosphate buffered saline (PBS, 1×) was obtained from Thermo Fisher Scientific, Waltham, Mass.

Bioactive Glass A (BG-A) was obtained from the 3M Corporation (Maplewood, Minn.) as a powder with the following composition: Na₂O (9.3 weight %), CaO (19.7 weight %), P₂O₅ (71.0 weight %). The reported glass transition temperature (Tg) was 418.5° C.
Bioactive Glass B (BG-B) was obtained from the 3M Corporation as a powder with the following composition: SiO₂ (45 weight %), Na₂O (24.5 weight %), CaO (24.5 weight %), P₂O₅ (6 weight %). The reported glass transition temperature (Tg) was 528.6° C.
Bioactive Glass C (BG-C) was obtained from the 3M Corporation as a powder with the following composition: Na₂O (5.5 weight %), CaO (18.5 weight %), P₂O₅ (3.7 weight %), K₂O (11.1 weight %), MgO (4.6 weight %), B₂O₃ (56.6 weight %). The reported glass transition temperature (Tg) was 518.8° C.

### Examples 1-9. Preparation of Collagen-Bioactive Glass Matrix

Collagen type I from bovine calf skin was dissolved in 20 mM acetic acid at a concentration of 5 mg/mL. A 15 mL aliquot of the solution was mixed with 200 mg, 400 mg, or 800 mg of either Bioactive Glass A, Bioactive Glass B, or Bioactive Glass C. Mixing was done using a SPEEDMIXER DAC 150.1 FVZ (obtained from FlackTek Incorporated, Landrum, S.C.) set at 3500 rpm (revolutions per minute) for 30 seconds. The resulting suspension was cast into a 6.3 cm inner diameter plastic tray (pre-treated with Rocket Release #E302 food grade release agent, obtained from Stoner Molding Solutions, Quarryville, Pa.) and then frozen at negative 20° C. for at least one hour. The frozen suspension was lyophilized to provide the collagen-bioactive glass product as a conformable, porous matrix. The products had variable thicknesses of about 1-7 mm. Lyophilizations were conducted using a VirTis Advantage Plus EL-85 Freeze Dryer (SP Scientific, Warminster, Pa.).

Conductivity measurements of the lyophilized products were conducted according to described Method A. Each lyophilized sample was weighed and immersed in distilled water at a concentration of 10 mg/mL. Each sample was maintained immersed in the water for 16 hours and then the conductivity (mS/cm) of the water was measured using a VWR SYMPHONY pH\conductivity meter (model# B30PCI, obtained from the VWR Corporation, Radnor, Pa.). The results are presented in Table 1.

TABLE 1
Conductivity (Method A)

| | | Weight of Matrix | |
|---|---|---|---|
| | | Sample after | |
| | Bioactive Glass in | Lyophilization | Conductivity |
| | Matrix Sample | (mg) | (mS/cm) |
| Control | None (Matrix Sample | NA | 0.121 |
| | Not Included) | | |
| Example 1 | BG-A (200 mg) | 238 | 1.03 |
| Example 2 | BG-A (400 mg) | 318 | 1.07 |
| Example 3 | BG-A (800 mg) | 360 | 1.10 |
| Example 4 | BG-B (200 mg) | 303 | 1.42 |
| Example 5 | BG-B (400 mg) | 397 | 1.72 |
| Example 6 | BG-B (800 mg) | 337 | 1.14 |
| Example 7 | BG-C (200 mg) | 320 | 2.42 |
| Example 8 | BG-C (400 mg) | 423 | 1.91 |
| Example 9 | BG-C (800 mg) | 447 | 2.15 |

### Examples 10-13

Collagen type I from bovine calf skin was dissolved in 20 mM acetic acid at a concentration of 5 mg/mL. A 20 mL aliquot of the solution was mixed with 200 mg or 400 mg of either Bioactive Glass A, Bioactive Glass B, or Bioactive Glass C. Comparative Example A was also prepared with no bioactive glass included in the sample. Mixing was done using a SPEEDMIXER DAC 150.1 FVZ (obtained from FlackTek Incorporated) set at 3500 rpm (revolutions per minute) for 30 seconds. Each suspension sample was transferred to a glass vial and allowed to settle for 20 minutes at room temperature. Sample viscosities were measured using a Brookfield Viscometer DV2T with an RV-06 spindle (obtained from AMETEK Brookfield, Middleboro, Mass.) at room temperature (23° C.). Viscosity measurements were taken at instrument settings of 25 rpm, 50 rpm, 100 rpm, and 200 rpm. The measured viscosities (cP) are reported in Table 2.

The pH of the samples was measured using a VWR SYMPHONY pH\conductivity meter and the results are presented in Table 3. The viscosity and pH measurements were performed within 1 hour of sample preparation.

TABLE 2
Viscosity Measurement

| | | Viscosity (Cp) | | | |
|---|---|---|---|---|---|
| | Bioactive Glass in | 25 | 50 | 100 | 200 |
| | Suspension Sample | rpm | rpm | rpm | rpm |
| Comparative | None | 200 | 160 | 130 | 105 |
| Example A | | | | | |
| Example 10 | BG-B (200 mg) | 1120 | 400 | 170 | 80 |
| Example 11 | BG-B (400 mg) | 880 | 240 | 80 | 80 |
| Example 12 | BG-A (200 mg) | 240 | 140 | 80 | 50 |
| Example 13 | BG-C (200 mg) | NT* | NT* | NT* | NT* |

NT = Not Tested
*The sample could not be tested for viscosity, because it instantly formed a gel.

TABLE 3
pH Measurement

| | Bioactive Glass in | |
|---|---|---|
| | Suspension Sample | pH |
| Comparative | None | 4.4 |
| Example A | | |
| Example 10 | BG-B (200 mg) | 9.4 |
| Example 11 | BG-B (400 mg) | 9.8 |
| Example 12 | BG-A (200 mg) | 4.9 |
| Example 13 | BG-C (200 mg) | 8.3 |

### Example 14. Collagen-Bioactive Glass Films Containing Glycerol

Glycerol (200 microliters) was added to 20 mL of a solution of collagen type I from bovine calf skin (5 mg/mL concentration) in 20 mM acetic acid. Next, 200 mg of a bioactive glass (selected from either Bioactive Glass A, Bioactive Glass C, or Bioactive Glass C) was added to the solution. The mixture was mixed using a SPEEDMIXER DAC 150.1 FVZ (obtained from FlackTek Incorporated) set at 3500 rpm for 30 seconds. Each suspension was then cast into a separate 6 plastic tray (6.3 cm diameter, pre-treated with Rocket Release #E302 food grade release agent, obtained from Stoner Molding Solutions) and then air dried at room temperature for 16 hours. Each dried product was removed from the tray in the form of a conformable, elastic, semi-transparent thin film.

### Example 15. Effect of Collagen-Bioactive Glass Matrix on pH

Collagen-bioactive glass matrix samples of Examples 1-9 were prepared. Comparative Example B was also prepared according to the procedure of Examples 1-9 with no bioactive glass included in the sample. The samples were tested for pH according to Method B (described above). Each lyophilized sample was weighed and placed in a separate 50 mL plastic tray. Distilled water (pH 6.8-7.2) was added to the tray so that the concentration of immersed sample was 10 mg/mL of liquid. The samples were maintained immersed in the liquid for 24 hours. The pH of the liquid in each tray was measured at the timepoints of 20 minutes and 24 hours after sample immersion. A VWR SYMPHONY pH\conductivity meter (model# B30PCI) was used to measure the pH. The results are presented in Table 4.

TABLE 4
pH Measurements using Distilled Water (method B)

| | Bioactive Glass in | pH at | pH at |
|---|---|---|---|
| | Matrix Sample | 20 Minutes | 24 Hours |
| Comparative | None | 6.55 | 6.99 |
| Example B | | | |
| Example 1 | BG-A (200 mg) | 7.0 | 6.71 |
| Example 2 | BG-A (400 mg) | 6.64 | 6.42 |
| Example 3 | BG-A (800 mg) | 6.54 | 6.27 |
| Example 4 | BG-B (200 mg) | 9.44 | 9.97 |
| Example 5 | BG-B (400 mg) | 9.70 | 10.51 |
| Example 6 | BG-B (800 mg) | 10.07 | 10.90 |
| Example 7 | BG-C (200 mg) | 9.37 | 9.61 |
| Example 8 | BG-C (400 mg) | 9.48 | 9.68 |
| Example 9 | BG-C (800 mg) | 9.52 | 9.72 |

### Example16. Effect of Collagen-Bioactive Glass Matrix on pH

The same procedure to test for pH as describe in Example 15 was followed with the exception that phosphate buffered saline (PBS, 1×) was used as the pH testing liquid, instead of distilled water. The results are presented in Table 5.

TABLE 5
pH Measurements using Phosphate Buffered Saline Solution

| | Bioactive Glass in | pH at | pH at |
|---|---|---|---|
| | Matrix Sample | 20 Minutes | 24 Hours |
| Comparative | None | 6.94 | 6.98 |
| Example B | | | |
| Example 1 | BG-A (200 mg) | 7.18 | 6.61 |
| Example 2 | BG-A (400 mg) | 6.96 | 6.53 |
| Example 3 | BG-A (800 mg) | 6.38 | 6.35 |
| Example 4 | BG-B (200 mg) | 8.26 | 10.59 |
| Example 5 | BG-B (400 mg) | 8.94 | 11.03 |
| Example 6 | BG-B (800 mg) | 9.23 | 11.54 |
| Example 7 | BG-C (200 mg) | 9.07 | 9.38 |
| Example 8 | BG-C (400 mg) | 9.09 | 9.46 |
| Example 9 | BG-C (800 mg) | 9.16 | 9.49 |

All references and publications cited herein are expressly incorporated herein by reference in their entirety into this disclosure. Illustrative embodiments of this invention are discussed and reference has been made to possible variations within the scope of this invention. For example, features depicted in connection with one illustrative embodiment may be used in connection with other embodiments of the invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. An article comprising,
a conformable matrix comprising a mixture of collagen and bioactive glass;
wherein the article is a conformable wound dressing.

2. The article of claim 1, wherein the article comprises a substrate.

3. The article of claim 1, wherein the conformable matrix has a conductivity of less than 3 mS/cm as determined by Method A.

4. The article of claim 1, wherein the conformable matrix is disposed on or within a carrier.

5. The article of claim 1, wherein the conformable matrix has a pH value more than 5.

6. The article of claim 1, wherein the conformable matrix further comprises glycerol.

7. The article of claim 1, wherein the conformable matrix is a gel, sponge, scaffold, foam or film.

8. A method of making an article, comprising
dissolving collagen in an acid solution;
adding bioactive glass to the acid solution;
mixing collagen and bioactive glass to increase or decrease the pH of the solution; and
forming a mixture of collagen and bioactive glass.

9. The method of claim 8, further comprising forming the mixture into a gel, sheet, film, sponge, foam, or a plurality of pieces.

10. The method of claim 8, further comprising dehydrating the mixture.

11. The method of claim 8, wherein the dehydrating comprises freeze-drying, oven drying, critical point drying, or combination thereof.

12. The method of claim 8, further comprising adding the mixture on a substrate.

13. The method of claim 8, wherein the article is a conformable wound dressing.

14. The method of claim 8, wherein the mixture of collagen and bioactive glass has a less than 3 mS/cm conductivity determined by Method A.

15. The method of claim 8, further comprising disposing the mixture on or within a carrier.
